(19) United States
(12) Patent Application Publication
(10) Pub. No.: US 2002/0115159 A1
(43) Pub. Date: Aug. 22, 2002

(54) Nucleotide sequences coding for the ATR61protein

(75) Inventors: Mike Farwick, Bielefeld (DE); Klaus Huthmacher, Gelnhausen (DE); Walter Pfefferle, Halle (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 09/953,259

(22) Filed: Sep. 17, 2001

(30) Foreign Application Priority Data
Sep. 15, 2000 (DE) 100 45 579.4

(51) Int. Cl. C12P 13/08; C12Q 1/68; C07H 21/04; C12N 1/21; C12P 21/02

(52) U.S. Cl. 435/115; 435/69.1; 435/6; 435/252.3; 435/252.33; 536/23.2

(57) ABSTRACT

The invention provides nucleotide sequences from Coryneform bacteria which code for the Atr61 protein and a process for the fermentative preparation of amino acids using bacteria in which the atr61 gene is enhanced.

### CROSS-REFERENCE TO RELATED APPLICATIONS

The present application claims priority to German Application No. DE 100 45 579.4, which was filed on Sep. 15, 2000, the entire contents of which is incorporated herein by reference.

### BACKGROUND OF THE INVENTION

1. Field of the Invention

The invention provides nucleotide sequences from Coryneform bacteria which code for the Atr61 protein and a process for the fermentative preparation of amino acids using bacteria in which the atr61 gene is enhanced.

2. Discussion of the Background

L-Amino acids, in particular L-lysine, are used in human medicine, pharmaceuticals, and foodstuffs, particularly in animal nutrition.

Fermentative preparation of amino acids from strains of Coryneform bacteria, in particular Corynebacterium glutamicum have been previously described. Because of their great importance, work is constantly being undertaken to improve these preparation processes. Improvements often relate to fermentation measures, such as, stirring and supply of oxygen; the composition of the nutrient media, such as, the sugar concentration during fermentation; the work-up of the amino acid by, for example, ion exchange chromatography; or by modifying the intrinsic output properties of the microorganism itself.

To effectuate the output properties of microorganims, mutagenesis and mutant selection are used. Strains which are resistant to antimetabolites or are auxotrophic for metabolites of regulatory importance and produce amino acids may be obtained in this manner.

Recombinant DNA techniques have also been employed to improve amino acid production, for example by modifying the strain of amplifying individual amino acid biosynthesis genes and investigating the effect on the amino acid production.

However, there remains a critical need for improved methods of producing L-amino acids and thus for the provision of strains of bacteria producing higher amounts of L-amino acids. On a commercial or industrial scale even small improvements in the yield of L-amino acids, or the efficiency of their production, are economically significant. Prior to the present invention, it was not recognized that enhancing the atr61 gene encoding the ABC transporter would improve L-amino acid yields.

### SUMMARY OF THE INVENTION

An object of the present invention is to provide novel measures for the improved production of L-amino acids or amino acid, where these amino acids include L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan, L-arginine and the salts (monohydrochloride or sulfate) thereof.

One object of the present invention is providing a novel process for improving the fermentative production of said L-amino acids, particularly L-lysine. Such a process includes enhanced bacteria, preferably enhanced Coryneform bacteria, which express enhanced amounts of a Atr61 ABC transporter protein or protein that has Atr61 protein activity.

Thus, another object of the present invention is providing such a bacterium, which expresses enhanced amounts of a Atr61 protein or gene products of the atr61 gene.

Another object of the present invention is providing a bacterium, preferably a Coryneform bacterium, which expresses a polypeptide that has enhnaced Atr61 protein activity.

Another object of the invention is to provide a nucleotide sequence encoding a polypeptide having the Atr61 protein sequence. One embodiment of such a sequence is the nucleotide sequence of SEQ ID NO: 1.

A further object of the invention is a method of making Atr61 protein or an isolated polypeptide having the activity of the Atr61 ABC transporter protein, as well as use of such isolated polypeptides in the production of amino acids. One embodiment of such a polypeptide is the polypeptide having the amino acid sequence of SEQ ID NO: 2.

Other objects of the invention include methods of detecting nucleic acid sequences homologous to SEQ ID NO: 1, particularly nucleic acid sequences encoding polypeptides that have the ABC transporter, and methods of making nucleic acids encoding such polypeptides.

The above objects highlight certain aspects of the invention. Additional objects, aspects and embodiments of the invention are found in the following detailed description of the invention.

### BRIEF DESCRIPTION OF THE DRAWINGS

FIG. 1: Map of the plasmid pEC-XK99E

FIG. 2: Map of the plasmid pEC-XK99Eatr6lex.

### DETAILED DESCRIPTION OF THE INVENTION

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Reference is made to standard textbooks of molecular biology that contain definitions and methods and means for carrying out basic techniques, encompassed by the present invention. See, for example, Sambrook et al., Molecular Cloning: A Laboratory Manual, Cold Spring Harbor Laboratory, New York (1989), Current Protocols in Molecular Biology, Ausebel et al (eds), John Wiley and Sons, Inc. New York (2000)and the various references cited therein.

“L-amino acids” or “amino acids” as used herein mean one or more amino acids, including their salts, chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-Lysine is particularly preferred. Preferred saltsinclude the monochlorides and sulfates.

The invention provides an isolated polynucleotide from Coryneform bacteria, comprising a polynucleotide sequence which codes for the atr61 gene, chosen from the group consisting of

a) polynucleotide which is identical to the extent of at least 70% to a polynucleotide which codes for a polypeptide which comprises the amino acid sequence of SEQ ID No. 2,

b) polynucleotide which codes for a polypeptide which comprises an amino acid sequence which is identical to the extent of at least 70% to the amino acid sequence of SEQ ID No. 2,

c) polynucleotide which is complementary to the polynucleotides of a) or b), and

d) polynucleotide comprising at least 15 successive nucleotides of the polynucleotide sequence of a), b) or c),

the polypeptide preferably having the activity of the ABC transporter Atr61.

The invention also provides the above-mentioned polynucleotide, this preferably being a DNA which is capable of replication, comprising:

(i) the nucleotide sequence shown in SEQ ID No. 1, or

(ii) at least one sequence which corresponds to sequence (i) within the range of the degeneration of the genetic cede, or

(iii) at least one sequence which hybridizes with the sequence complementary to sequence (i) or (ii), and optionally

(iv) sense mutations of neutral function in (i).

The invention also provides

a polynucleotide, in particular DNA, which is capable of replication and comprises the nucleotide sequence as shown in SEQ ID No. 1;

a polynucleotide which codes for a polypeptide which comprises the amino acid sequence as shown in SEQ ID No. 2;

a vector containing the polynucleotide according to the invention, in particular a shuttle vector or plasmid vector, and

Coryneform bacteria which contain the vector or in which the endogenous atr61 gene is enhanced.

The invention also provides polynucleotides which substantially comprise a polynucleotide sequence, which are obtainable by screening by means of hybridization of a corresponding gene library of a Coryneform bacterium, which comprises the complete gene or parts thereof, with a probe which comprises the sequence of the polynucleotide according to the invention according to SEQ ID No. 1 or a fragment thereof, and isolation of the polynucleotide sequence mentioned.

Polynucleotides which comprise the sequences according to the invention are suitable as hybridization probes for RNA, cDNA and DNA, in order to isolate, in the full length, nucleic acids or polynucleotides or genes which code for the ABC transporter Atr61 or to isolate those nucleic acids or polynucleotides or genes which have a high similarity of sequence with that of the atr61 gene.

Additionally, methods employing DNA chips, microarrays or similar recombinant DNA technology that enables high throughput screening of DNA and polynucleotides which encode the Atr61 protein or polynucleotides with homology to the atr61 gene as described herein. Such methods are known in the art and are described, for example, in Current Protocols in Molecular Biology, Ausebel et al (eds), John Wiley and Sons, Inc. New York (2000).

Polynucleotides which comprise the sequences according to the invention are furthermore suitable as primers with the aid of which DNA of genes which code for the ABC transporter Atr61 can be prepared by the polymerase chain reaction (PCR).

Such oligonucleotides which serve as probes or primers comprise at least 25, 26, 27, 28, 29 or 30, preferably at least 20, 21, 22, 23 or 24, more preferably at least 15, 16, 17, 18 or 19 successive nucleotides. Oligonucleotides which have a length of at least 31, 32, 33, 34, 35, 36, 37, 38, 39 or 40, or at least 41, 42, 43, 44, 45, 46, 47, 48, 49 or 50 nucleotides are also suitable.

Oligonucleotides with a length of at least 100, 150, 200, 250 or 300 nucleotides are optionally also suitable.

“Isolated” means separated out of its natural environment.

“Polynucleotide” in general relates to polyribonucleotides and polydeoxyribonucleotides, it being possible for these to be non-modified RNA or DNA or modified RNA or DNA.

The polynucleotides according to the invention include a polynucleotide according to SEQ ID No. 1 or a fragment prepared therefrom and also those which are at least in particular 70% to 80%, preferably at least 81% to 85%, more prefered at least 86% to 90%, and very particularly preferably at least 91%, 93%, 95%, 97% or 99% identical to the polynucleotide according to SEQ ID No. 1 or a fragment prepared therefrom.

“Polypeptides” are understood as meaning peptides or proteins which comprise two or more amino acids bonded via peptide bonds.

The polypeptides according to the invention include a polypeptide according to SEQ ID No. 2, in particular those with the biological activity of the ABC transporter Atr61, and also those which are at least 70% to 80%, preferably at least 81% to 85%, particularly preferably at least 86% to 90%, and more prefered at least 91%, 93%, 95%, 97% or 99% identical to the polypeptide according to SEQ ID No. 2 and have the activity mentioned.

The invention furthermore relates to a process for the fermentative preparation of amino acids chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine using Coryneform bacteria which in particular sequences which code for the atr61 gene are enhanced, in particular over-expressed.

The term “enhancement” in this connection describes the increase in the intracellular activity of one or more enzymes in a microorganism which are coded by the corresponding DNA, for example by increasing the number of copies of the gene or allele or of the genes or alleles, using a potent promoter or using a gene or allele which codes for a corresponding enzyme (protein) having a high activity, and optionally combining these measures.

By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on that of the wild-type protein or the activity or concentration of the protein in the starting microorganism.

The microorganisms which the present invention provides can produce L-amino acids from glucose, sucrose, lactose, fructose, maltose, molasses, starch, cellulose or from glycerol and ethanol. They can be representatives of Coryneform bacteria, in particular of the genus Coryneform. Of the genus Coryneform, there may be mentioned in particular the species Corynebacterium glutamicum, which is known among experts for its ability to produce L-amino acids.

Suitable strains of the genus Corynebacterium, in particular of the species Corynebacterium glutamicum (C. glutamicum), are in particular the known wild-type strains

Corynebacterium glutamicum ATCC13032

Corynebacterium acetoglutamicum ATCC15806

Corynebacterium acetoacidophilum ATCC 13870

Corynebacterium thermoaminogenes FERM BP-1539

Corynebacterium melassecola ATCC 17965

Brevibacterium flavum ATCC 14067

Brevibacterium lactofermentum ATCC13869 and

Brevibacterium divaricatum ATCC14020

and L-amino acid-producing mutants or strains prepared therefrom.

Preferably, a bacterial strain with enhanced expression of a atr61 gene that encodes a polypeptide with Atr61 ABC transporter activity will improve amino acid yield at least 1%.

The new atr61 gene from C. glutamicum which codes for the ABC transporter Atr61 has been isolated.

To isolate the atr61 gene or also other genes of C. glutamicum, a gene library of this microorganism is first set up in Escherichia coli (E. coli). The setting up of gene libraries is described in generally known textbooks and handbooks. The textbook by Winnacker: Gene and Klone, Eine Einfuhrung in die Gentechnologie [Genes and Clones, An Introduction to Genetic Engineering] (Verlag Chemie, Weinheim, Germany, 1990), or the handbook by Sambrook et al.: Molecular Cloning, A Laboratory Manual (Cold Spring Harbor Laboratory Press, 1989) may be mentioned as an example. A well-known gene library is that of the E. coli K-12 strain W3110 set up in k vectors by Kohara et al. (Cell 50, 495-508 (1987)). Bathe et al. (Molecular and General Genetics, 252:255-265, 1996) describe a gene library of C. glutamicum ATCC 13032, which was set up with the aid of the cosmid vector SuperCos I (Wahl et al., 1987, Proceedings of the National Academy of Sciences USA, 84:2160-2164) in the E. coli K-12 strain NM554 (Raleigh et al., 1988, Nucleic Acids Research 16:2563-1575).

Bormann et al. (Molecular Microbiology 6(3), 317-326) (1992)) in turn describe a gene library of C. glutamicum ATCC13032 using the cosmid pHC79 (Hohn and Collins, Gene 11, 291-298 (1980)).

To prepare a gene library of C. glutamicum in E. coli it is also possible to use plasmids such as pBR322 (Bolivar, Life Sciences, 25, 807-818 (1979)) or pUC9 (Vieira et al., 1982, Gene, 19:259-268). Suitable hosts are, in particular, those E. coli strains which are restriction- and recombination-defective. An example of these is the strain DHSamcr, which has been described by Grant et al. (Proceedings of the National Academy of Sciences USA, 87 (1990) 4645-4649). The long DNA fragments cloned with the aid of cosmids can in turn be subcloned in the usual vectors suitable for sequencing and then sequenced, as is described e.g. by Sanger et al. (Proceedings of the National Academy of Sciences of the United Status of America, 74:5463-5467, 1977).

The resulting DNA sequences can then be investigated with known algorithms or sequence analysis programs, such as e. g, that of Staden (Nucleic Acids Research 14, 217-232(1986)), that of Marck (Nucleic Acids Research 16, 1829-1836 (1988)) or the GCG program of Butler (Methods of Biochemical Analysis 39, 74-97 (1998)).

The new DNA sequence of C. glutamicum which codes for the atr61 gene and which, as SEQ ID No. 1, is a constituent of the present invention has been found. The amino acid sequence of the corresponding protein has furthermore been derived from the present DNA sequence by the methods described above. The resulting amino acid sequence of the atr61 gene product is shown in SEQ ID No. 2.

Coding DNA sequences which result from SEQ ID No. 1 by the degeneracy of the genetic code are also a constituent of the invention. In the same way, DNA sequences which hybridize with SEQ ID No. 1 or parts of SEQ ID No. 1 are a constituent of the invention. Conservative amino acid exchanges, such as e.g. exchange of glycine for alanine or of aspartic acid for glutamic acid in proteins, are furthermore known among experts as “sense mutations” which do not lead to a fundamental change in the activity of the known that changes on the N and/or C terminus of a protein cannot substantially impair or can even stabilize the function thereof. Information in this context can be found by the expert, inter alia, in Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in known textbooks of genetics and molecular biology. Amino acid sequences which result in a corresponding manner from SEQ ID No. 2 are also a constituent of the invention.

In the same way, DNA sequences which hybridize with SEQ ID No. 1 or parts of SEQ ID No. 1 are a constituent of the invention. Finally, DNA sequences which are prepared by the polymerase chain reaction (PCR) using primers which result from SEQ ID No. 1 are a constituent of the invention. Such oligonueleotides typically have a length of at least 15 nucleotides.

Instructions for identifying DNA sequences by means of hybridization can be found by the expert, inter alia, in the handbook “The DIG System Users Guide for Filter Hybridization” from Boehringer Mannheim GmbH (Mannheim, Germany, 1993) and in Liebl et al. (International Journal of Systematic Bacteriology (1991) 41: 255-260). The hybridization takes place under stringent conditions, that is to say only hybrids in which the probe and target sequence, i.e. the polynucleotides treated with the probe, are at least 70% identical are formed. It is known that the stringency of the hybridization, including the washing steps, is influenced or determined by varying the buffer composition, the temperature and the salt concentration. The hybridization reaction is preferably carried out under a relatively low stringency compared with the washing steps (Hybaid Hybridisation Guide, Hybaid Limited, Teddington, UK, 1996).

A 5×SSC buffer at a temperature of approx. 50° C.-68° C., for example, can be employed for the hybridization reaction. Probes can also hybridize here with polynucleotides which are less than 70% identical to the sequence of the probe. Such hybrids are less stable and are removed by washing under stringent conditions. This can be achieved, for example, by lowering the salt concentration to 2×SSC and optionally subsequently 0.5×SSC (The DIG System User's Guide for Filter Hybridisation, Boehringer Mannheim, Mannheim, Germany, 1995) a temperature of approx. 50° C.-68° C. being established. It is optionally possible to lower the salt concentration to 0.1×SSC. Polynucleotide fragments which are, for example, at least 70% or at least 80% or at least 90% to 95% identical to the sequence of the probe employed can be isolated by increasing the hybridization temperature stepwise from 50° C. to 68° C. in steps of approx. 1-2° C. Further instructions on hybridization can be obtained by known methods in the art, e.g., as a described in Sambrook et al or in kits readily available in the art, e.g. DIG Easy Hyb from Roche Diagnostics GmbH, Mannheim, Germany, Catalogue No. 1603558).

Instructions for amplification of DNA sequences with the aid of the polymerase chain reaction (PCR) can be found by the expert, inter alia, in the handbook by Gait: Oligonucleotide Synthesis: A Practical Approach (IRL Press, Oxford, UK, 1984) and in Newton and Graham: PCR (Spektrum Akademischer Verlag, Heidelberg, Germany, 1994).

It has been found that Coryneform bacteria produce amino acids in an improved manner after over-expression of the atr61 gene.

To achieve an over-expression, the number of copies of the corresponding genes can be increased, or the promoter and regulation region or the ribosome binding site upstream of the structural gene can be mutated. Expression cassettes which are incorporated upstream of the structural gene act in the same way. By inducible promoters, it is additionally possible to increase the expression in the course of fermentative amino acid production. The expression is likewise improved by measures to prolong the life of the mRNA. Furthermore, the enzyme activity is also increased by preventing the degradation of the enzyme protein. The. genes or gene constructs can either be present in plasmids with a varying number of copies, or can be integrated and amplified in the chromosome. Alternatively, an over-expression of the genes in question can furthermore be achieved by changing the composition of the media and the culture procedure.

Instructions in this context can be found by the expert, inter alia, in Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in EP 0 472 869, in U.S. Pat. No. 4,601,893, in Schwarzer and Puhler (Bio/Technology 9, 84-87 (1991), in Reinscheid et al. (Applied and Environmental Microbiology 60, 126-132 (1994)), in LaBarre et al. (Journal of Bacteriology 175, 1001-1007 (1993)), in WO 96/15246, in Malumbres et al. (Gene 134, 15-24 (1993)), in JP-A-10-229891, in Jensen and Hammer (Biotechnology and Bioengineering 58, 191-195 (1998)), in Makrides (Microbiological Reviews 60:512-538 (1996)) and in known textbooks of genetics and molecular biology.

By way of example, for enhancement the atr61 gene according to the invention was over-expressed with the aid of episomal plasmids. Suitable plasmids are those which are replicated in Coryneform bacteria. Numerous known plasmid vectors, such as e.g. pZ1 (Menkel et al., Applied and Environmental Microbiology (1989) 64: 549-554), pEKEx1 (Eikmanns et al., Gene 102:93-98 (1991)) or pHS2-1 (Sonnen et al., Gene 107:69-74 (1991)) are based on the cryptic plasmids pHM 15 19, pBL 1 or pGA1. Other plasmid vectors, such as e.g. those based on pCG4 (U.S. Pat. No. 4,489,160), or pNG2 (Serwold-Davis et al., FEMS Microbiology Letters 66, 119-124 (1990)), or pAGI (U.S. Pat. No. 5,158,891), can be used in the same manner.

Plasmid vectors which are furthermore suitable are also those with the aid of which the process of gene amplification by integration into the chromosome can be used, as has been described, for example, by Reinscheid et al. (Applied and Environmental Microbiology 60, 126-132 (1994)) for duplication or amplification of the hom-thrB operon. In this method, the complete gene is cloned in a plasmid vector which can replicate in a host (typically E. coli), but not in C. glutamicum. Possible vectors are, for example, pSUP301 (Simon et al., Hio/Technology 1, 784-791 (1983)), pKI8mob or pKl9mob (Schafer et al., Gene 145, 69-73 (1994)), pGEM-T (Promega Corporation, Madison, Wis., USA), pCR2. 1-TOPO (Shuman (1994)). Journal of Biological Chemistry 269:32678-84; U.S. Pat. No. 5,487,993), pCR®Blunt (Invitrogen, Groningen, Holland; Bernard et al., Journal of Molecular Biology, 234: 534-541 (1993)), pEMI (Schrumpf et al, 1991, journal of Bacteriology 173:4510-4516) or pBGS8 (Spratt et al.,1986, Gene 41: 337-342). The plasmid vector which contains the gene to be amplified is then transferred into the desired strain of C. glutamicum by conjugation or transformation. The method of conjugation is described, for example, by Schafer et al. (Applied and Environmental Microbiology 60, 756-759 (1994)). Methods for transformation are described, for example, by Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross over” event, the resulting strain contains at least two copies of the gene in question.

In addition, it may be advantageous for the production of L-amino acids to enhance, in particular over-express one or more enzymes of the particular biosynthesis pathway, of glycolysis, of anaplerosis, of the citric acid cycle, of the pentose phosphate cycle, of amino acid export and optionally regulatory proteins, in addition to the atr61 gene.

Thus, for the preparation of L-amino acids, in addition to enhancement of the atr6l gene, one or more endogenous genes chosen from the group consisting of

the dapA gene which codes for dihydrodipicolinate synthase (EP-B 0 197 335),

the gap gene which codes for glyceraldehyde 3-phosphate dehydrogenase (Eikmanns (1992), Journal of Bacteriology 174:6076-6086),

the tpi gene which codes for triose phosphate isomerase (Eikmanns (1992), Journal of Bacteriology 174:6076-6086),

the pgk gene which codes for 3-phosphoglycerate kinase (Eikmanns (1992), Journal of Bacteriology 174:6076-6086),

the zwf gene which codes for glucose 6-phosphate dehydrogenase (JP-A-09224661),

the pyc gene which codes for pyruvate carboxylase (DE-A-198 31 609),

the mqo gene which codes for malate-quinone oxidoreductase (Malenaar et al., European Journal of Biochemistry 254, 395-403 (1998)),

the lysC gene which codes for a feed-back resistant aspartate kinase (Accession No. P26512; EP-B-0387527; EP-A-0699759),

the lysE gene which codes for lysine export (DE-A-195 48 222),

the hom gene which codes for homoserine dehydrogenase (EP-A 0131171),

the ilvA gene which codes for threonine dehydratase (Mockel et al., Journal of Bacteriology (1992) 8065-8072)) or the ilvA(Fbr) allele which codes for a “feed back resistant” threonine dehydratase (Mockel et al., (1994) Molecular Microbiology 13: 833-842),

the ilvBN gene which codes for acetohydroxy-acid synthase (EP-B 0356739),

the ilvD gene which codes for dihydroxy-acid dehydratase (Sahm and Eggeling (1999) Applied and Environmental Microbiology 65: 1973-1979),

the zwal gene which codes for the Zwal protein (DE: 19959328.0, DSM 13115), can be enhanced, in particular over-expressed.

It may furthermore be advantageous for the production of L-amino acids, in addition to the enhancement of the atr61 gene, for one or more genes chosen from the group consisting of:

the pck gene which codes for phosphoenol pyruvate carboxykinase (DE 199 50 409.1; DSM 13047),

the pgi gene which codes for glucose 6-phosphate isomerase (U.S. Pat. No. 09/396,478; DSM 12969),

the poxB gene which codes for pyruvate oxidase (DE: 1995 1975.7; DSM 13114),

the zwa2 gene which codes for the Zwa2 protein (DE: 19959327.2, DSM 13113)

to be attenuated, in particular for the expression thereof to be reduced.

The term “attenuation” in this connection describes the reduction or elimination of the intracellular activity of one or more enzymes (proteins) in a microorganism which are coded by the corresponding DNA, for example by using a weak promoter or using a gene or allele which codes for a corresponding enzyme with a low activity or inactivates the corresponding gene or enzyme (protein), and optionally combining these measures.

By attenuation measures, the activity or concentration of the corresponding protein is in general reduced to 0 to 75%, 0 to 50%, 0 to 25%, 0 to 10% or 0 to 5% of the activity or concentration of the wild-type protein or of the activity or concentration of the protein in the starting microorganism.

In addition to over-expression of the atr61 gene it may furthermore be advantageous for the production of amino acids to eliminate undesirable side reactions (Nakayama: “Breeding of Amino Acid Producing Micro-organisms”, in: Overproduction of Microbial Products, Krumphanzl, Sikyta, Vanek (eds.), Academic Press, London, UK, 1982).

The invention also provides the microorganisms prepared according to the invention, and these can be cultured continuously or discontinuously in the batch process (batch culture) or in the fed batch (feed process) or repeated fed batch process (repetitive feed process) for the purpose of production of amino acids. A summary of known culture methods is described in the textbook by Chmiel (Bioprozesstechnik 1. Einfuhrung in die Bioverfahrenstechnik [Bioprocess Technology 1. Introduction to Bioprocess Technology (Gustav Fischer Verlag, Stuttgart, 1991)) or in the textbook by Storhas (Bioreaktoren and periphere Einrichtungen [Bioreactors and Peripheral Equipment] (Vieweg Verlag, Braunschweig/Wiesbaden, ].999)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981).

Sugars and carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substance can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as e. g. magnesium sulfate or iron sulfate, which are necessary for growth. Finally, essential growth substances, such as amino acids and vitamins, can be employed in addition to the above-mentioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, such as e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 20° C. to 45° C., and preferably 25° C. to 40° C. Culturing is continued until a maximum of the desired product has formed. This target is usually reached within 10 hours to 160 hours.

Methods for the determination of L-amino acids are known from the prior art. The analysis can thus be carried out, for example, as described by Spackman et al. (Analytical Chemistry, 30, (1958), 1190) by ion exchange chromatography with subsequent ninhydrin derivation, or it can be carried out by reversed phase HPLC, for example as described by Lindroth et al. (Analytical Chemistry (1979) 51: 1167-1174).

The process according to the invention is used for fermentative preparation of amino acids.

The following microorganism was deposited as a pure culture on Aug. 22, 2001 at the Deutsche Sammlung fur Mikroorganismen and Zellkulturen (DSMZ=German Collection of Microorganisms and Cell Cultures, Braunschweig, Germany) in accordance with the Budapest Treaty:

Escherichia coli DH5alphamcr/pEC-XK99Eatr6lex (=DH5amcr/pEC-XK99Eatr6lex) as DSM 14461.

The present invention is explained in more detail in the following with the aid of embodiment examples.

The isolation of plasmid DNA from Escherichia coil and all techniques of restriction, Klenow and alkaline phosphatase treatment were carried out by the method of Sambrook et al. (Molecular Cloning. A Laboratory Manual (1989) Cold Spring Harbor Laboratory Press, Cold Spring Harbor, N.Y., USA). Methods far transformation of Escherichia coli are also described in this handbook.

The composition of the usual nutrient media, such as LB or TY medium, can also be found in the handbook by Sambrook et al.

Having generally described this invention, a further understanding can be obtained by reference to certain specific examples which are provided herein for purposes of illustration only, and are not intended to be limiting unless otherwise specified.

### EXAMPLES

### Example 1

Preparation of a genomic cosmid gene library from Corynebacterium glutamicum ATCC 13032

Chromosomal DNA from Corynebacterium glutamicum ATCC 13032 was isolated as described by Tauch et al. (1995, Plasmid 33:168-179) and partly cleaved with the restriction enzyme Sau3AI (Amersham Phsrmacia, Freiburg, Germany, Product Description Sau3AI, Code no. 27-0913-02). The DNA fragments were dephosphorylated with shrimp alkaline phosphatase (Roche Diagnostics GmbH, Mannheim, Germany, Product Description SAP, Code no. 1758250). The DNA of the cosmid vector SuperCosl (Wahl et al. (1987) Proceedings of the National Academy of Sciences USA 84:2160-2164), obtained from Stratagene (La Jolla, USA, Product Description SuperCosl Cosmid Vector Kit, Code no. 251301) was cleaved with the restriction enzyme XbaI (Amersham Pharmacia, Freiburg, Germany, Product Description XbaI, Code no. 27-0948-02) and likewise dephosphorylated with shrimp alkaline phosphatase.

The cosmid DNA was then cleaved with the restriction enzyme BamHI (Amersham Pharmacia, Freiburg, Germany, Product Description BamHI, Code no. 27-0868-04). The cosmid DNA treated in this manner was mixed with the treated ATCC13032 DNA and the batch was treated with T4 DNA ligase (Amersham Pharmacia, Freiburg, Germany, Product Description T4-DNA-Ligase, Code no. 27-0870-04). The ligation mixture was then packed in phages with the aid of Gigapack II XL Packing Extract (Stratagene, La Jolla, USA, Product Description Gigapak II XL Packing Extract, Code no. 200217).

For infection of the E. coli strain NM554 (Raleigh et al. 1988, Nucleic Acid Research 16:1563-1575) the cells were taken up in 10 mM MgSO₄ and mixed with an aliquot of the phage suspension. The infection and titering of the cosmid library were carried out as described by Sambrook et al. (1989, Molecular Cloning: A laboratory Manual, Cold Spring Harbor), the cells being plated out on LB agar (Lennox, 1955, Virology, 1:190) with 100 mg/l ampicillin. After incubation overnight at 37° C., recombinant individual clones were selected.

### Example 2

Isolation and sequencing of the atr61 gene

The cosmid DNA of an individual colony was isolated with the Qiaprep Spin 1:5-Miniprep Kit (Product No. 27106, Qiagen, Hil den, Germany) in accordance with the manufacturer's instructions and partly cleaved with the restriction enzyme Sau3AI (Amersham Pharmacia, Freiburg, Germany, Product Description Sau3AI, Product No. 27-0913-02). The DNA fragments were dephosphorylated with shrimp alkaline phosphatase (Roche Diagnostics GmbH, Mannheim, Germany, Product Description SAP, Product No. 20 1758250). After separation by gel electrophoresis, the cosmid fragments in the site range of 1500 to 2000 bp were isolated with the QiaExII Gel Extraction Kit (Product No. 2002 1, Qiagen, Hilden, Germany).

The DNA of the sequencing vector pZero-1, obtained, from Invitrogen (Groningen, Holland, Product Description Zero Background Cloning Kit, Product No. K2500-01), was cleaved with the restriction enzyme BamHI (Amersham Pharmacia, Freiburg, Germany, Product Description BamHI, Product No. 27-0868-04). The ligation of the cosmid fragments in the sequencing vector pZero-1 was carried out as described by Sambrook et al. (1989, Molecular Cloning: A Laboratory Manual, Cold Spring Harbor), the DNA mixture being incubated overnight with T4 ligase (Pharmacia Biotech, Freiburg, Germany). This ligation mixture was then electroporated (Tauch et al. 1994, FEMS Microbiol Letters, 123:343-7) into the E. coli strain DH5αMCR (Grant, 1990, Proceedings of the National Academy of Sciences U.S.A., 87:4645-4649) and plated out on LB agar (Lennox, 1955, Virology, 1:190) with 50 mg/l zeocin.

The plasmid preparation of the recombinant clones was carried out with the Biorobot 9600 (Product No. 900200, Qiagen, Hilden, Germany). The sequencing was carried out by the dideoxy chain termination method of Sanger et al. (1977, Proceedings of the National Academy of Sciences U.S.A., 74:5463-5467) with modifications according to Zimmermann et al. (1990, Nucleic Acids Research, 18:1067). The “R R dRhodamin Terminator Cycle Sequencing Kit” from P E Applied Biosystems (Product No. 403044, Weiterstadt, Germany) was used. The separation by gel electrophoresis and analysis of the sequencing reaction were carried out in a “Rotiphoresis NF Acrylamide/Bisacrylamide” Gel (29: 1) (Product No. A124.1, Roth, Karlsruhe, Germany) with the “ABI Prism 377” sequencer from P E Applied Biosystems (Weiterstadt, Germany).

The raw sequence data obtained were then processed using the Staden program package (1986, Nucleic Acids Research, 14:217-231) version 97-0. The individual sequences of the pZerol derivatives were assembled to a continuous contig. The computer-assisted coding region analysis was prepared with the XNIP program (Staden, 1986, Nucleic Acids Research, 14:217-231).

The resulting nucleotide sequence is shown in SEQ ID No. 1. Analysis of the nucleotide sequence showed an open reading frame of 1866 base pairs, which was called the atr61 gene. The atr61 gene codes for a protein of 621 amino acids.

### Example 3

Preparation of a shuttle vector pXK99Eatr6lex for enhancement of the atr61 gene in C. glutamicum

3.1 Cloning of the atr61 gene in the vector pCR®Blunt II

From the strain ATCC 13032, chromosomal DNA was isolated by the method of 10 Eikmanns et al. (Microbiology 140: 1817-1828 (1994)). On the basis of the sequence of the atr61 gene known for C. glutamicum from example 2, the following oligonucleotides were chosen for the polymerase chain reaction (see SEQ ID No. 3 and SEQ ID No. 4):

atr6lex1:

5′-ct ggtacc—cac cac cta cta atg cga ct-3′

atr6Iex2:

5′ga tctaga—ggg cta gtc ctc ttc ttc ag-3′

The primers shown were synthesized by MWG-Biotech AC (Ebersberg, Germany) and the PCR reaction was carried out by the standard PCR method of Innis et al. (PCR Protocols. A Guide to Methods and Applications, 1990, Academic Press) with Pwo-Polymerase from Roche Diagnostics GmbH (Mannheim, Germany). With the aid of the polymerase chain reaction, the primers allow amplification of a DNA fragment 1897 bp in size, which carries the atr61 gene. Furthermore, the primer atr61 ex1 contains the sequence for the cleavage site of the restriction endonuclease Kpnl, and the primer atr61ex2 the cleavage site of the restriction endonuclease XbaI, which are marked by underlining in the nucleotide sequence shown above.

The atr61 fragment 1897 bp in size was cleaved with the restriction endonucleases KpnI and XbaI and then isolated from the agarose gel with the QiaExII Gel Extraction Kit (Product No. 20021, Qiagen, Hilden, Germnany).

3.2 Construction of the shuttle vector pEG,XK99E

The E. coli—C. glutamicum shuttle vector pEC-XK99E was constructed according to the prior art. The vector contains the replication region rep of the plasmid pGA1 including the replication effector per (U.S. Pat. No. 5,175,108; Nesvera et al., Journal of Bacteriology 179, 1525-1532 (1997)), the kanamycin resistance gene aph(3′)-IIa from Escherichia coli (Beck et al. (1982), Gene 19: 327-336), the replication origin of the trc promoter, the termination regions T1 and T2, the lacI^{q} gene (repressor of the lac operon of E. coli) and a multiple cloning site (mcs) (Norrander, J. M. et al. Gene 26, 101-106 (1983)) of the plasmid pTRC99A (Amann et al. (1988), Gene 69: 301-315).

The trc promoter can be induced by addition of the lactose derivative IPTG (isopropyl β-D-thiogalactopyranoside).

The E. coli—C. glutamicum shuttle vector pEC-XK99E constructed was transferred into C. glutamicum DSM5715 by means of electroporation (Liebl et al., 1989, FEMS Microbiology Letters, 53:299-303). Selection of the transformants took place on LBHIS agar comprising 18.5 g/l brain-heart infusion broth, 0.5 M sorbitol, 5 g/1 Bactotryptone, 2.5 g/l Bacto-yeast extract, 5 g/l NaCI and 18 g/l Bacto-agar, which had been supplemented with 25 mg/1 kanamycin. Incubation was carried out for 2 days at 33° C.

Plasmid DNA was isolated from a transformant by conventional methods (Peters-Wendisch et al., 1998, Microbiology, 144, 915 -927), cleaved with the restriction endonuclease HindlIl, and the plasmid was checked by subsequent agarose gel electrophoresis.

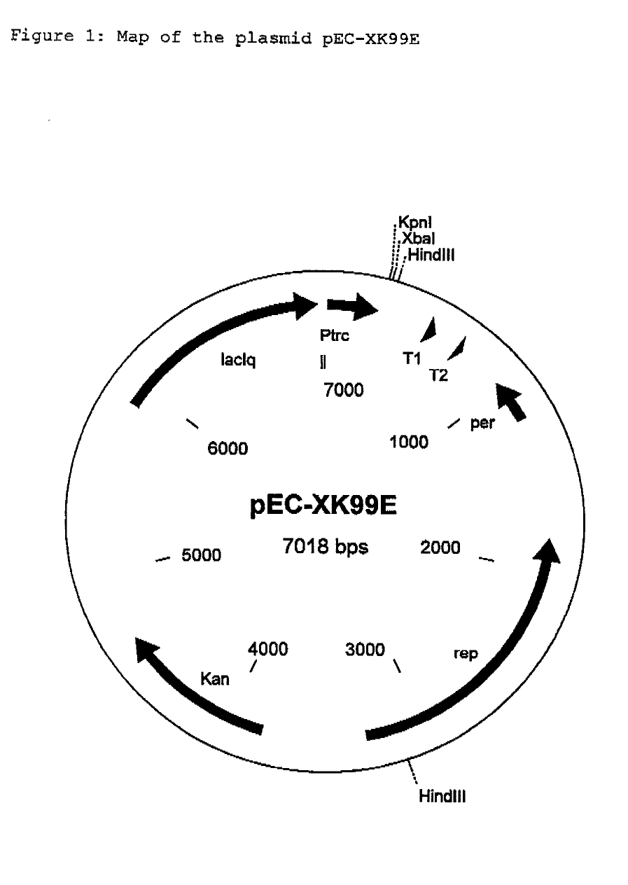

The plasmid construct obtained in this way was called pEC-XK99E (FIG. 1). The strain obtained by electroporation of the plasmid pEC-XK99E in the C. glutamicum strain DSM5715 was called DSM5715/pEC-XK99E and deposited as DSM13455 at the Deutsche Sammlung fur Mikroorganismen and Zellkulturen (DSMZ=German Collection of Microorganisms and Cell Cultures, Braunschweig, Germany) in accordance with the Budapest Treaty.

3.3 Cloning of atr61 in the E. coli—C. glutamicum shuttle vector pEC-XK99E

The E. coli—C. glutamicum shuttle vector pEC-XK99E described in example 3.2 was used as the vector. DNA of this plasmid was cleaved completely with the restriction enzymes KpnI and Xbal and then dephosphorylated with shrimp alkaline phosphatase (Roche Diagnostics GmbH, Mannheim, Germany, Product Description SAP, Product No. 1758250).

The atr61 fragment approx. 1880 bp in size described in example 3.1, obtained by means of PCR and cleaved with the restriction endonucleases Kpnl and Xbal was mixed with the prepared vector pEC-XK99E and the batch was treated with T4 DNA ligase (Amersham Pharmacia, Freiburg, Germany, Product Description T4-DNA-Ligase, Code no.27-0870-04). The ligation batch was transformed in the E. coli strain DH5 amcr (Hanahan, In: DNA cloning. A Practical Approach. Vol.1I, IRL-Press, Oxford, Washington DC, USA). Selection of plasmid-carrying cells was made by plating out the transformation batch an LB agar (Lennox, 1955, Virology, 1: 190) with 50 mg/l kanamycin. After incubation overnight at 37° C., recombinant individual clones were selected.

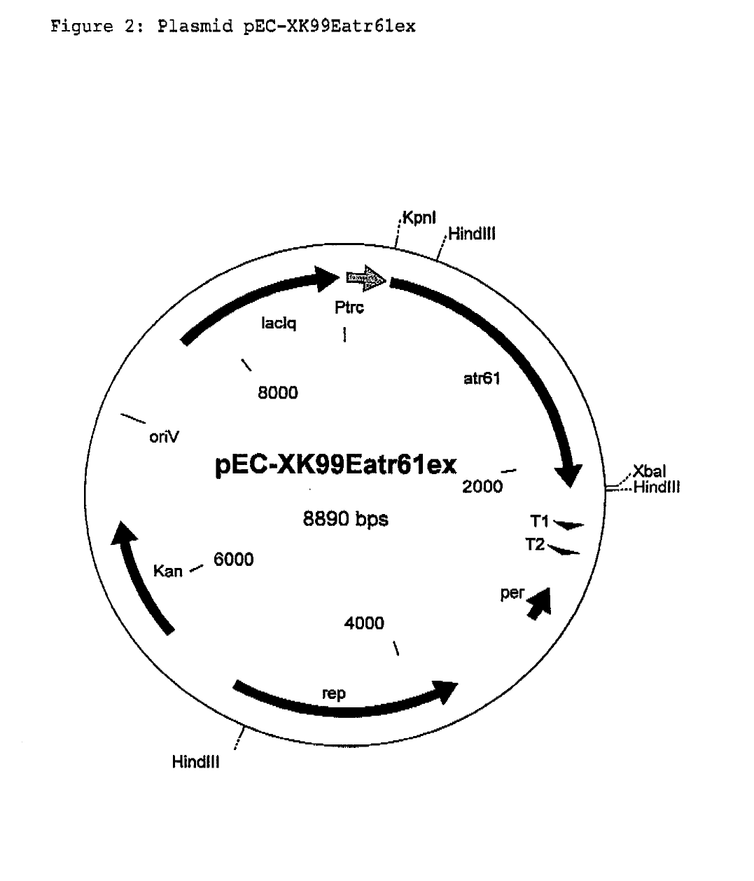

Plasmid DNA was isolated from a transformant with the Qiaprep Spin Miniprep Kit (Product No. 27106, Qiagen, Hilden, Germany) in accordance with the manufacturer's instructions and cleaved with the restriction enzymes XbaI and KpnI to check the plasmid by subsequent agarose gel electrophoresis. The resulting plasmid was called pEC-XK99atr6lex. It is shown in FIG. 2.

The abbreviations and designations used have the following meaning:

| | |
|---|---|
| Kan: | Kanamycin resistance gene aph(3′)-11a from Escherichia coli |
| HindIIl | Cleavage site of the restriction enzyme HindIII |
| XbaI | Cleavage site of the restriction enzyme XbaI |
| KpnI | Cleavage site of the restriction enzyme Kpnl |
| Ptrc | trc promoter |
| T1 | Termination region T1 |
| T2 | Termination region T2 |
| per | Replication effector per |
| rep | Replication region rep of the plasmid pGAI |
| lacIq | laclq repressor of the lac operon of Escherichia coli |
| atr61 | Isolated atr61 gene |

Obviously, numerous modifications and variations on the present invention are possible in light of the above teachings. It is therefore to be understood that within the scope of the appended claims, the invention may be practiced otherwise than as specifically described herein.

What is claimed is:

1. An isolated polynucleotide, which encodes a protein having the amino acid sequence of SEQ ID NO:2.

2. The isolated polynucleotide of claim 1, wherein said protein has ABC transporter activity.

3. An isolated polynucleotide, which comprises SEQ ID NO: 1.

4. An isolated polynucleotide, which is complimentary to the isolated polynucleotide of claim 3.

5. An isolated polynucleotide, which is at least 70% identical to the polynucleotide of claim 3.

6. An isolated polynucleotide, which is at least 80% identical to the polynucleotide of claim 3.

7. An isolated polynucleotide, which is at least 90% identical to the polynucleotide of claim 3.

8. An isolated polynucleotide which hybridizes under stringent conditions to the polynucleotide of claim 3, wherein said stringent conditions comprise washing in 5×SSC at a temperature from 50 to 68° C.

9. The isolated polynucleotide of claim 3, which encodes a protein having ABC transporter activity.

10. An isolated polynucleotide, which comprises at least 15 consecutive nucleotides of the isolated polynucleotide of claim 3.

11. A vector comprising the isolated polynucleotide of claim 1.

12. A vector comprising the isolated polynucleotide of claim 3.

13. A host cell comprising the isolated polynucleotide of claim 1.

14. A host cell comprising the isolated polynucleotide of claim 3.

15. The host cell of claim 13, which is a Coryneform bacterium.

16. The host cell of claim 14, which is a Coryneform bacterium.

17. The host cell of claim 13, which is selected from the group consisting of Coryneform glutamicum, Corynebacterium acetoglutamicum, Corynebacterium acetoacidophilum, Corynebacterium thermoaminogenes, Corynebacterium melassecola, Brevibacterium flavum, Brevibacterium lactofermentum, and Brevibacterium divaricatum.

18. The host cell of claim 14, which is selected from the group consisting of Coryneform glutamicum, Corynebacterium acetoglutamicum, Corynebacterium acetoacidophilum, Corynebacterium thermoaminogenes, Corynebacterium melassecola, Brevibacterium flavum, Brevibacterium lactofermentum, and Brevibacterium divaricatum.

19. A Coryneform bacterium which comprises an enhanced atr61 gene.

20. The Coryneform bacterium of claim 19, wherein said atr61 gene comprises the sequence of SEQ ID NO: 1.

21. Escherichia coli DSM 14461.

22. A process for producing L-amino acids comprising culturing a bacterial cell in a medium suitable for producing L-amino acids, wherein said bacterial cell comprises an enhanced atr61 gene.

23. The process of claim 22, wherein said atr61 gene comprises SEQ ID NO: 1.

24. The process of claim 22, wherein said atr61 gene comprises a polynucleotide sequence which hybridizes under stringent conditions to the sequence of SEQ ID NO: 1, wherein said stringent conditions comprise washing in 5×SSC at a temperature of from 50 to 68° C.

25. The process of claim 24, wherein said polynucleotide which hybridizes under stringent conditions to the sequence of SEQ ID NO: 1, is at least 90% identical to SEQ ID NO:1.

26. The process of claim 22, wherein said bacterial cell is a Coryneform bacterium or Brevibacterium.

27. The process of claim 22, wherein said bacterial cell is selected from the group consisting of Coryneform glutamicum, Corynebacterium acetoglutamicum, Corynebacterium acetoacidophilum, Corynebacterium melassecola, Corynebacterium thermoaminogenes, Brevibacterium flavum, Brevibacterium lactofermentum, and Brevibacterium divaricatum.

28. The process of claim 22, wherein said L-amino acid is L-lysine.

29. The process of claim 22, wherein said bacteria further comprises at least one gene whose expression is enhanced, wherein said gene is selected from the group consisting of dapA4, gap, tpl, pgk, zwf, pyc, rnqo, lysC, lyse, horn, ilvA, ilvBN, ilvD and zwa 1.

30. The process of claim 22, wherein said bacteria further comprises at least one gene whose expression is attenuated, wherein said gene is selected from the group consisting of pck, pgi, poxB, and zwa2.

31. A process for screening for polynucleotides, which encode a protein having ABC transporter activity comprising
a. hybridizing the isolated polynucleotide of claim 1 to the polynucleotide to be screened;
b. expressing the polynucleotide to produce a protein; and
c. detecting the presence or absence of ABC transporter activity in said protein.

32. A process for screening for polynucleotides, which encode a protein having ABC transporter activity comprising
a. hybridizing the isolated polynucleotide of claim 3 to the polynucleotide to be screened;
b. expressing the polynucleotide to produce a protein; an d
c. detecting the presence or absence of ABC transporter activity in said protein.

33. A method for detecting a nucleic acid with at least 70% homology to nucleotide of claim 3, comprising contacting a nucleic acid sample with a probe or primer comprising at least 15 consecutive nucleotides of the nucleotide sequence of claim 3, or at least 15 consecutive nucleotides of the complement thereof.

34. A method for producing a nucleic acid with at least 80% homology to nucleotide of claim 3, comprising contacting a nucleic acid sample with a primer comprising at least 15 lo3 a consecutive nucleotides of the nucleotide sequence of claim 3, or at least 15 consecutive nucleotides of the complement thereof.

35. A method for screening for polynucleotides which encode a protein having ABC transporter activity comprising
a. hybridizing the isolated polynucleotide of claim 1 to the polynucleotide to be screened;
b. expressing the polynucleotide to produce a protein; and
c. detecting the presence or absence of ABC transporter activity in said protein.

36. A method for making a Atr61 protein, comprising
a. culturing the host cell of claim 13 for a time and under conditions suitable for expression of the Atr61 protein; and
b. collecting the Atr61 protein.

37. A method for making a Atr61 protein, comprising
a. culturing the host cell of claim 14 for a time and under conditions suitable for expression of the Atr61 protein; and
b. collecting the Atr61 protein.

38. An isolated polypeptide comprising the amino acid sequence of SEQ ID NO:2.


SEQUENCE LISTING (4 sequences)

SEQ ID NO: 1 - DNA, 2320 bp, Corynebacterium glutamicum
  CDS (251)..(2113)
ggtgaaaagcgggcacagaatcagccgggcacaacgagggatatgtatcccaactggtgtatcccactgtgtgacagcgaaggcaactccgtgctcattgaatcgctgcgtgaaaatgagctgtatcaccgtgtggcaaaggcaagcaagcgagattaggtccgcttcagttgtggtggctccgaatctgatgaacaatgatcattcctaattcatttacatctttatcaaagagagccaccacctactaatgcgacttcttggtcgaattttaaaaaccacgtctgcgctttggccctactatctcggaattatcgtcgtatccattgtgatcgcggcgttgtcgctgctgtcgccgtttattctccgcgaagcaacagattccattgtttctgcagtaaccggatctaacaccgtcgatgcagttactcgcactattattttcttagctttagccctgtttgtcgcaagcttcctcaatacggtgatgaccaacatcggtggctacatcggtgatgtcatggcatctcgtatgcgccagattctggccacgcgctattacgcaaagctgttggcgctgcctcagaagtattttgataatcaggtcaccggcaccatcatcgcccgccttgatcgatcaatcaacggcatcacgcagttcatgcagagcttctccaacaacttcttccccatgctcatcaccatggtggcagtgctgattatttccgcgattttctactggcctctggcaattctgctggccatgttgttcccgatttacatgtggctgacggcgttgacatcgaaacgctggcagaaatatgagggcgagaaaaaccatgaaatcgacgtggctaacggccgcttcgctgaggttgtcggccaggtcaaggttgttaaatcattcgtcgcagagacccgcgagctggctgatttcggtgggcgttacggcaaaacagtagcgattacccggccgcaatccggttggtggcaccgcatggatactctccgtggcgcggcactaaatatcatcttcctggccattcacctgctgattttctaccgcaccttgcacggccatttcaccatcggcgacatggtcatgctcatccagcttgtcaccatggcgcagcaaccggtgtacatgatgagctacatcgtcgactccgcgcagcgcgccatcgccggctcccgcgactacttcgaggtcatggcgcagcaggtcgagcccaccgccaataaggagcttgtcgacgccaccctcgcctcagacactccacgcatcagtgtgggcacgcccgccgcgctgcccgctggagaaccagcgatggaattcaaaaacgtcaccttcgcctacgaagaaggcaagccggttatttccgacgtgtccattaccgcccgccacggcgagcgcatcgcgttggtcggtgaatccggcggcggtaaatccaccctggtcaaccttctgttaggtctgtacaaaccaaacagcggcagccttgcagtatgtggcgtggatgttaaagatctgacttccgaggaacttcgcgcatccgtgggtgtggtcttccaggacgccagcttgttctctggatctattgcagaaaacatcgcctacggtcgcccaggtgccacccgcgaagagatcatcgaagtggctaagaaagccaacgcacatgagttcatttccgccttccctgaaggatatgaaaccgtcgtcggtgaacgcggactcaaactttctggtggccagaagcagcgcgtctctgtggcacgggccatgcttaaagatgccccacttcttgttctcgatgaagccacctctgcactggataccaagtctgagcaggcagtccaagccggtttggaacagctgatggaaaaccgcaccaccttaatgatcgcccaccgcctgtccaccatcgcaggcgtcgataccatcgtgaccatccaaaacggacgggttgaagaggtcggatctcctaccgagctcgcagtctcaggcggtatctattccgaactgctgcgcctgaccaactccacagcagaagccgaccgggagcgtctgcgcgcctttggtttcactggcgatgcaccagctgaagaagaggactagccccgcgaaagaacaatcccccagtgcccaaaggtaccaggggatttttcttaattccatgctctagatcaaggaaacaagccacgcaataacaacgatgaccagcaacaccacgagcgcttgggtaacgcgtgactgtgggtacttgcgcttacgcttcgggagttcttggtctctcttgcggagctcttctgggttagccat

SEQ ID NO: 2 - protein, 621 aa, Corynebacterium glutamicum
MRLLGRILKTTSALWPYYLGIIVVSIVIAALSLLSPFILREATDSIVSAVTGSNTVDAVTRTIIFLALALFVASFLNTVMTNIGGYIGDVMASRMRQILATRYYAKLLALPQKYFDNQVTGTIIARLDRSINGITQFMQSFSNNFFPMLITMVAVLIISAIFYWPLAILLAMLFPIYMWLTALTSKRWQKYEGEKNHEIDVANGRFAEVVGQVKVVKSFVAETRELADFGGRYGKTVAITRPQSGWWHRMDTLRGAALNIIFLAIHLLIFYRTLHGHFTIGDMVMLIQLVTMAQQPVYMMSYIVDSAQRAIAGSRDYFEVMAQQVEPTANKELVDATLASDTPRISVGTPAALPAGEPAMEFKNVTFAYEEGKPVISDVSITARHGERIALVGESGGGKSTLVNLLLGLYKPNSGSLAVCGVDVKDLTSEELRASVGVVFQDASLFSGSIAENIAYGRPGATREEIIEVAKKANAHEFISAFPEGYETVVGERGLKLSGGQKQRVSVARAMLKDAPLLVLDEATSALDTKSEQAVQAGLEQLMENRTTLMIAHRLSTIAGVDTIVTIQNGRVEEVGSPTELAVSGGIYSELLRLTNSTAEADRERLRAFGFTGDAPAEEED

SEQ ID NO: 3 - DNA, 28 bp, Artificial Sequence
  synthetic DNA
ctggtacccaccacctactaatgcgact

SEQ ID NO: 4 - DNA, 28 bp, Artificial Sequence
  synthetic DNA
gatctagagggctagtcctcttcttcag